(19) United States
(12) Patent Application Publication
(10) Pub. No.: US 2012/0160723 A1
(43) Pub. Date: Jun. 28, 2012

(54) Fixing Means

(75) Inventors: Michael Harms, Frankfurt am Main (DE); Steffen Raab, Frankfurt am Main (DE); Udo Stauder, Frankfurt am Main (DE); Volker Korger, Frankfurt am Main (DE); Uwe Dasbach, Frankfurt am Main (DE); Caroline Stephan, Offenbach (DE)

(73) Assignee: SANOFI-AVENTIS DEUTSCHLAND GMBH, Frankfurt am Main (DE)

(21) Appl. No.: 13/254,096

(22) PCT Filed: Mar. 31, 2010
(86) PCT No.: PCT/EP10/54351

§ 371 Date: Feb. 29, 2012

Related U.S. Application Data
(60) Provisional application No. 61/169,877, filed on Apr. 16, 2009.

(30) Foreign Application Priority Data
Mar. 31, 2009 (EP) 09004673.1

(51) Int. Cl. A61M 5/00; B65D 25/10; F16L 3/08

(52) U.S. Cl. 206/438; 248/74.2

(57) ABSTRACT

A fixing means (1) for a drug delivery device (6) comprises at least two clip elements (2) for releasably securing at least one drug delivery device (6) to said fixing means (1), wherein each of the two clip elements (2) comprises a flexible holding member (3) and wherein the clip elements (2) share one flexible holding member (4) with each other.

This disclosure relates to a fixing means for a drug delivery device.

Patients using drug delivery devices often have to dispense regularly a dose of a drug, for example insulin, for guaranteeing an adequate supply with said drug. Hence, users often have to carry said drug delivery devices with them.

It is an object of the present disclosure to provide for a means which facilitates a safe storage and/or a safe transportation of a drug delivery device.

This object may be achieved by a fixing means according to the independent claim. Further features and advantageous embodiments are subject matter of the dependent claims.

According to one aspect a fixing means for a drug delivery device is provided. The fixing means may comprise at least one clip element for releasably recurring at least one drug delivery device to the fixing means. The clip element may be adapted and arranged to releasably secure the drug delivery device to the fixing means.

The drug delivery device may be a pen-type device, e.g. a pen-type injector. The drug delivery device may comprise a cartridge containing a drug.

When having delivered a dose of the drug, a user can attach the drug delivery device to the fixing means, thereby securing the device in the at least one clip element of the fixing means. Hence, the drug delivery device may be secured against inadvertent linear movement and/or rotation with respect to the fixing means. If required, the user may remove the device quickly from the fixing means and administer a(nother) dose of the drug.

In a preferred embodiment the clip element is configured to encompass the at least one drug delivery device.

Preferably, the clip element comprises a shape, which is configured to adapt optimally to an outer surface of the drug delivery device.

In a preferred embodiment, the at least one clip element comprises two holding members which are arranged to receive and retain the at least one drug delivery device in a holding space extending between the holding members. The respective holding member may have a free end.

Preferably, the clip element comprises two holding members which may adapt to a shape of the device, preferably encompassing said device, hence reliably securing the drug delivery device to the fixing means.

According to a preferred embodiment, at least one of the two holding members, preferably both, are flexible.

Preferably, the two holding members are made of a flexible material for facilitating securing the device to and unsecuring the device from the fixing means. The flexible material of the flexible holding members may be an elastomer or any other flexible, preferably elastically, deformable material which facilitates the (un)securing of the drug delivery device.

According to a preferred embodiment, one of the two holding members is flexible and the other holding member is rigid.

Preferably, one of the two holding members is more rigid than the other (flexible) holding member. Preferably, the more rigid holding member is made of a material, which is not easily elastically deformable. Additionally or alternatively, the more rigid holding member may be broadened in comparison to the other (flexible) holding member.

According to a further preferred embodiment the at least one flexible holding member of the at least one clip element is configured to bow radially outwardly with respect to the at least one drug delivery device while introducing the device into or releasing the device from the fixing means.

A distance between the free ends of the holding members may be increased when securing or unsecuring the drug delivery device. One of the holding members or both of the holding members may be moved with respect to the other one of the holding members by bending one of or both of the holding members.

When the drug delivery device is secured to or unsecured from the fixing means, user-exerted force is transferred to the at least one clip element via the drug delivery device. The at least one flexible holding member of the clip element may bow radially outwardly when the force is transferred to it. This guarantees an easy and quick release of the device from the fixing means as well as a simple and reliable fixing of the device to the fixing means. Preferably, the at least one flexible holding member is configured to bow radially inwardly back into a starting position once the drug delivery device has been fully guided through the intermediate space between the free ends into the clip element. Accordingly, the two holding members may encompass the device, thus, the drug delivery device may be secured to the fixing means.

In a preferred embodiment, the holding members comprise free ends and the distance between the free ends of the two holding members is smaller than the diameter of the drug delivery device which is to be received and, in particular, retained between the two holding members.

This embodiment facilitates reliable securing the device in the clip element. Once the device is retained in the clip element the at least one flexible holding member bows radially inwardly. The holding members preferably touch the drug delivery device (only) at their free ends. Accordingly, the holding members encompass the drug delivery device and secure the device to the fixing means.

According to another preferred embodiment, the fixing means comprises at least two clip elements.

For allowing for securing two drug delivery devices to the fixing means and therefore, to protect them from inadvertent movement with respect to each other and to the fixing means as well as from vibrations or other influences, which may damage the drug delivery devices said fixing means preferably comprises two clip elements. Preferably, the two clip elements are (firmly) connected with each other. The two clip elements may be formed unitarily. For securing more than two drug delivery devices to the fixing means the fixing means may contain three or more clip elements, which are preferably (firmly) connected with each other.

In another preferred embodiment, each of the two clip elements comprises one flexible member and the clip elements share one flexible or rigid holding member with each other. This facilitates a compact embodiment of a fixing means for two drug delivery devices.

In another preferred embodiment, the two clip elements are marked differently.

Two different drug delivery devices may be secured to the fixing means, e.g. devices containing different drugs, such as a long-acting or a short-acting insulin. The devices may be not easily distinguishable, such as by a differently designed exterior shape, for example. Therefore, it may be useful to design the two clip elements differently. This may help to distinguish the two different drug delivery devices more easily from one another and to prevent a confusion as to which drug delivery device contains which drug.

According to a preferred embodiment the two clip elements are marked with different colours.

Preferably, the two clip elements have different colours for distinguishing between the two different drug delivery devices secured to the fixing means. Therefore, a user can easily distinguish the drug delivery devices from each other and a confusion—which may have fatal and lethal consequences—may be prevented.

According to a preferred embodiment, the fixing means comprises at least one attaching means for attaching the fixing means to a carry case.

Preferably, the fixing means comprises at least one element which allows an easy securing of the fixing means to a carry case. The carry case may provide an additional protection of the drug delivery device, especially from environmental influences.

According to a preferred embodiment the attaching means comprises at least one cut-out.

According to a preferred embodiment the fixing means is configured to be glued into the carry case.

This presents a cost effective manner to attach the fixing means to the carry case.

According to another preferred embodiment the at least one drug delivery device is a pen-type drug delivery device.

In one embodiment an assembly is provided comprising a drug delivery device secured to the fixing means.

According to a preferred embodiment a carry case is provided, wherein the fixing means is attached to the carry case. The carry case preferably comprises a retaining means for retaining information about the at least one drug delivery device and/or for retaining information related to a user of said drug delivery device.

Preferably, the fixing means is secured to the carry case which may protect the fixing means and, hence, the device secured to said fixing means from environmental influences. Preferably, the carry case is closeable, for example by means of a zip, to protect the drug delivery device. The information retained in the retaining means may help a user to determine the intervals in which a dose of the (respective) drug has to be administered. Additionally or alternatively, the information retained may help to identify the owner of the carry case in case the carry case was lost. According to a preferred embodiment the retaining means comprises a rigid box. According to another preferred embodiment the retaining means comprises a pocket.

According to a preferred embodiment, a fixing means for a drug delivery device is provided comprising at least one clip element for releasably securing at least one drug delivery device to the fixing means.

According to a preferred embodiment, a fixing means for a drug delivery device is provided comprising at least two clip elements for releasably securing at least one drug delivery device to said fixing means. Each of the two clip elements comprises a flexible holding member. The clip elements share one flexible holding member with each other.

By means of the at least one clip element a drug delivery device may be securable against unintentinal movement with respect to the fixing means. Slipping of the device out of the fixing means and, hence, damage of the device may be prevented in this way.

Further features and refinements become apparent from the following description of the exemplary embodiments in connection with the accompanying figures.

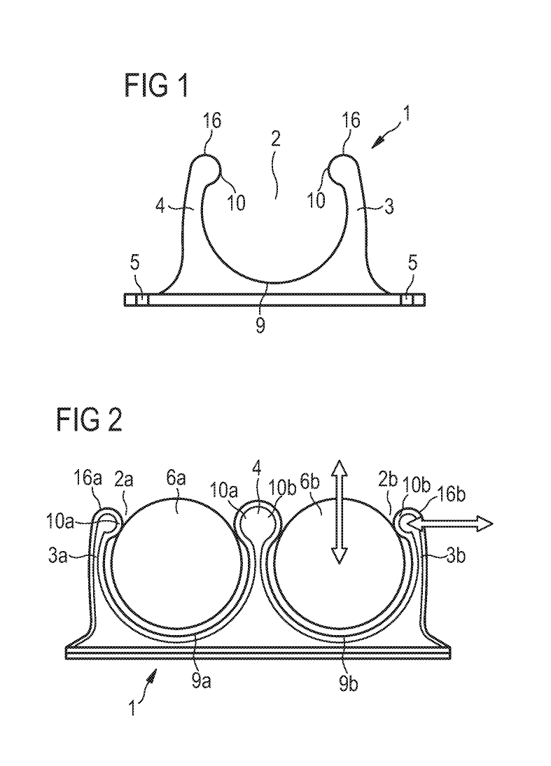

FIG. 1 schematically shows a fixing means according to a first embodiment on the basis of a sectional view,

FIG. 2 schematically shows a fixing means according to a second embodiment on the basis of a sectional view,

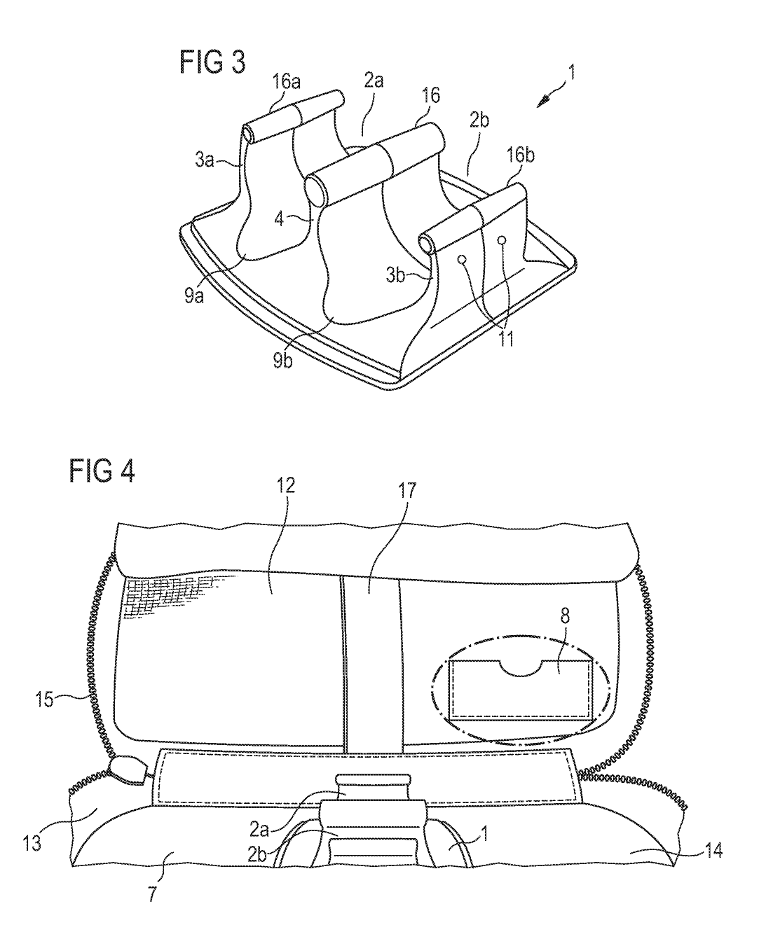

FIG. 3 schematically shows an oblique view onto a fixing means according to a third embodiment,

FIG. 4 schematically shows an embodiment of a carry case containing the fixing means of FIG. 2 on the basis of an oblique partial view,

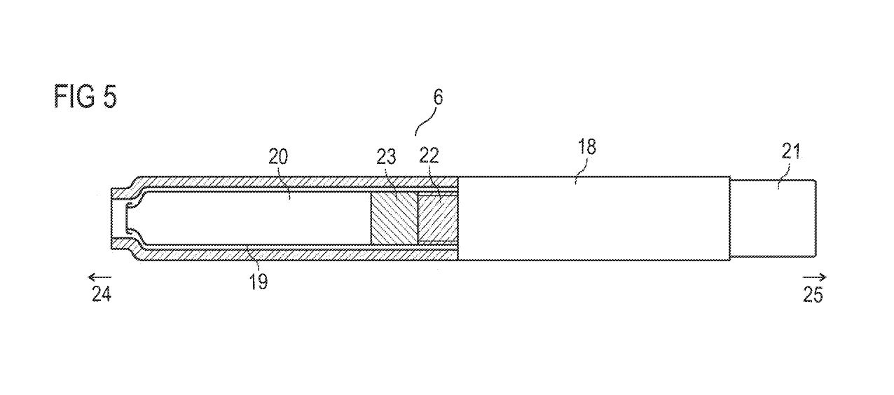

FIG. 5 schematically shows an embodiment of a drug delivery device which may be secured to a fixing means according to any of the previously described embodiments on the basis of a sectional view.

Like elements of the same kind and identically acting elements may be provided with the same reference numerals in the figures.

FIG. 1 schematically shows a fixing means 1, which comprises a clip element 2. The clip element 2 comprises two holding members 3, 4. The respective holding member 3, 4 or both holding members 3, 4 may be flexible fingers. The holding members 3, 4 comprise free ends 16 each comprising a bulge 10. The fixing means 1 further comprises attaching means 5. The clip element 2 comprises a bearing surface 9, which is positioned at a bottom of the clip element 2.

The fixing means 1 comprises the clip element 2, which releasably secures at least one drug delivery device (not shown in FIG. 1) to the fixing means 1. For this purpose, the clip element 2 may comprise the two holding members 3, 4, which delimit a holding space for a drug delivery device. The holding space is situated in-between the two holding members 3, 4. The holding members 3, 4 may encompass the drug delivery device (for details see FIG. 2) once the device has been introduced into the holding space and, in particular, bears on the bearing surface 9 of the clip element 2. Slipping of the drug delivery device out of the fixing means 1 and/or inadvertent movement of the drug delivery device with respect to the fixing means 1 may be prevented in this way.

One holding member or both holding members 3, 4 may be a flexible holding member 3, 4. Thus, one holding member 3 or 4 or both holding members 3, 4 may be made of a flexible material, for example, a plastic material, an elastomer or a thermoplast, which can be elastically deformed when securing a drug delivery device to or unsecuring said device from the fixing means 1. However, one or both holding members 3, 4 may also be made of any other flexible material, which can elastically deform considerably when force is transferred to them. Additionally or alternatively, one holding member 3, 4 may be more rigid than the other holding member 4 or 3. Additionally or alternatively, one holding member 3, 4 may be made of a material which is not easily elastically deformable when securing a drug delivery device to or unsecuring said device from the fixing means 1. Said material may be a rigid plastic, for example. Additionally or alternatively, one (more rigid) holding member 3, 4 may be broadened in comparison to the other holding member 4, 3 making said holding member 3, 4 more rigid than the other holding member 4, 3, in particular when the two holding members 3, 4 are made of the same material.

In the following both holding members 3, 4 are flexible holding members 3, 4. In this embodiment the two holding members 3, 4 are provided with the bulge 10 at their free end 16. The bulge 10 protrudes radially inwardly. The respective bulge 10 may abut the drug delivery device once the device was positioned on the bearing surface 9, as shown in FIG. 2 for example. The distance between the free ends 16 of the flexible holding members 3, 4 is smaller than the diameter of a drug delivery device which is to be placed or is placed between the two holding members 3, 4. This facilitates a save fixation of the device to the fixing means 1.

The two holding members 3, 4 receive the drug delivery device between each other and adapt due to the flexible embodiment to said device. Thereby, the outside of the drug delivery device abuts the free ends 16 of the holding members 3, 4 (see also FIG. 2), in particular the bulge 10 at the free ends 16 of the holding members 3, 4. Accordingly, the device is safely secured to the fixing means 1 and damaging of said device for example during transport can be prevented.

A user places the device on the free ends 16 of the holding members 3, 4 when securing the device to the clip element 2. Thereby, the user impacts a force which is directed towards the bearing surface 9 of the clip element 2. Hence, the two flexible holding members 3, 4 bow radially outwardly with respect to the drug delivery device. The device is then moved further towards the bearing surface 9. Once the device is positioned on the bearing surface 9 the flexible holding members 3, 4 bow radially inwardly with respect to the drug delivery device, preferably back into a starting position of the flexible holding members 3, 4 which the flexible holding members 3, 4 had before the drug delivery device was placed in the clip element 2. Due to the flexible embodiment of the two flexible holding members 3, 4 the device can be removed easily from the fixing means 1 and can be fixed easily to said fixing means 1 without a user having to apply an excessive force.

Once the device is secured to the fixing means 1, said fixing means 1 prevents a linear displacement and/or a rotation of the drug delivery device with respect to said fixing means 1. This facilitates a safe storage and/or a safe transport of the drug delivery device. Especially the latter may often be desirable, as a user may be forced to carry the drug delivery device with him in order to dispense a dose of drug at several, regular intervals during a day.

The fixing means 1 further comprises the attaching means 5, e.g. one or more cut-outs. In this embodiment two cut-outs are arranged laterally at the fixing means 1. By means of the attaching means 5, the fixing means 1 may be secured to a carry case (not shown in FIG. 1), which may provide additional protection of the drug delivery device from environmental influences, from vibrations or other effects which may damage the drug delivery device. Accordingly or alternatively, the fixing means 1 may be glued to the carry case. Alternatively or additionally, the fixing means 1 may comprise a groove (not explicitly shown in FIG. 1) which may run (circumferentially) along an outer edge of the fixing means 1, i.e. outside of the clip element 2. Preferably, the groove is arranged near an edge of the fixing means 1. The fixing means 1 may be sewed to the carry case already mentioned previously within that groove. Alternatively or additionally, the fixing means 1 may be secured to the carry case by means of a velcro connection.

Preferably, the fixing means 1 may be molded, for example from plastic or rubber. Thereby, the fixing means 1 may be integrally formed, and in particular, unitarily.

FIG. 2 schematically shows a fixing means 1 according to a second embodiment on the basis of a sectional view.

The fixing means 1 comprises two clip elements 2a, 2b. The two clip elements 2a, 2b each comprise holding members 3 (i.e. 3a and 3b) and 4. FIG. 2 further shows two drug delivery devices 6a, 6b.

As already mentioned in connection with FIG. 1, the fixing means 1 may be molded, for example from plastic or rubber. The fixing means 1 may be integrally formed.

The two clip elements 2a, 2b releasably secure the two drug delivery devices 6a, 6b to the fixing means 1. For this purpose, each clip element 2a, 2b comprises the two holding members 3a, 3b and 4, with the two clip elements 2a, 2b sharing one holding member 4 with each other. Hence, a compact embodiment of the fixing means 1 is facilitated.

The two clip elements 2a, 2b may be symmetrical or may comprise different sizes. One drug delivery device 6a, 6b is assigned to one clip element 2a, 2b, i.e. is placed in the holding space onto the bearing surface 9a, 9b of one clip element 2a, 2b. The holding space is surrounded by two holding members 3a, 3b and 4, respectively. The holding spaces of the two clip elements 2a, 2b are separated from each other by the shared holding member 4. The shared holding member 4 may be broadened in comparison to the holding members 3a, 3b. The shared holding member 4 comprises a bulge 10a, 10b at each side of its free end 16. The holding members 3a, 3b comprise one bulge 10a, 10b at their free end 16a, 16b, wherein the bulge 10a, 10b protrudes radially inwardly.

The two clip elements 2a, 2b may be symmetrical with respect to the shared holding member 4. Alternatively, in particular in case the two drug delivery device 6a, 6b comprise a different shape, the two clip elements 2a, 2b may comprise different sizes. In particular, the two clip elements 2a, 2b may define differently sized holding spaces between the holding members 3a, 3b and 4. The holding members 3a, 3b, 4 may be shaped differently. For example, the holding members 3a, 3b, 4 may comprise differently sized bulges 10, 10a, 10b at their free ends 16, 16a, 16b.

The holding members 3a, 3b and 4 of the two clip elements 2a, 2b may be made of a flexible material, for example a thermoplast, and hence bow radially outwardly, as indicated by the arrow pointing to the right, when the drug delivery device 6a, 6b is secured to or unsecured from the fixing means 1 (the movement of the drug delivery device 6a, 6b when secured to or unsecured from the fixing means 1 is indicated by the “up-down” arrow). Alternatively, only the two holding members 3a, 3b may be made of a flexible material. The shared holding member 4 is preferably broader than the two holding members 3a, 3b and may thus be less flexible than the holding members 3a, 3b. Additionally or alternatively, the shared holding member 4 may be made of a rigid material. Hence the holding member 4 may be an inflexible or rigid holding member. In the following the two holding members 3a, 3b may be flexible holding members and the shared holding member 4 may be a rigid holding member.

The flexible holding members 3a, 3b bow radially inwardly back into a starting position once the drug delivery device 6a, 6b has been guided through the holding space between the free ends 16a, 16b and 16, into the clip element 2a, 2b, as indicated by the arrow pointing to the left.

The drug delivery device 6a, 6b may be a pen-type injector, preferably a reusable injector or a disposable pre-filled injector. The drug delivery device 6a, 6b may be provided to dispense fixed doses of a drug or variable (preferably user-settable) doses.

The two clip elements 2a, 2b may be marked differently. For example, the two clip elements 2a, 2b may comprise different colours (not shown in FIG. 2) for fixing different drug delivery devices 6a, 6b to the fixing means 1. The different colours make it easier for a user to distinguish between the two different drug delivery devices 6a, 6b, above all if the devices 6a, 6b are of the same shape and/or size, but contain different drugs. Especially for diabetics, who often suffer from a strong amblyopia, different colours of the clip elements 2a, 2b may prevent the confusion of two different drug delivery devices 6a, 6b and hence, the administration of a dose of the wrong drug, which may, of course, have fatal consequences for the user.

The clip elements 2a, 2b may be distinguished from each other by tactile and/or visible (for example colour) markings. The clip elements 2a, 2b may comprise for example different shapes or sizes or differently configured surface structures, for example grooves or knobs (see knobs 11 in FIG. 3), or any other features which may facilitate distinguishing between two different drug delivery devices 6a, 6b. The respective markings of the clip element 2a, 2b should be visible when the drug delivery device 6a, 6b is secured to and when the device 6a, 6b not saved to the clip element 2a, 2b.

Alternatively, for securing more than two drug delivery devices 1 to the fixing means 1, the fixing means 1 may contain three or more clip elements 2.

Although not shown in FIG. 2, the fixing means 1 may again comprise attaching means 5 for securing the fixing means 1 to a carry case (see FIG. 1 and FIG. 4).

FIG. 3 schematically shows an oblique view of a fixing means 1 according to a third embodiment.

FIG. 3 represents a fixing means 1 comprising two clip elements 2a, 2b. The clip elements 2a, 2b comprise holding members 3a, 3b and 4. FIG. 3 also shows the bearing surfaces 9a, 9b of the clip elements 2a, 2b as well as knobs 11 on the surface of one clip element 2b, which may serve as markings as discussed above.

The fixing means 1 and the clip elements 2a, 2b may comprise the same features as already described in conjunction with FIG. 1 and FIG. 2. The clip elements 2a, 2b comprise once again two holding members 3a, 3b and 4 each, with the two clip elements 2a, 2b sharing the holding member 4. Thereby, in one embodiment the holding members 3a, 3b and 4 may again be made of a flexible material. In another embodiment, only the holding members 3a, 3b may be made of a flexible material, the shared and, in particular, broadened holding member 4 may be less flexible than the holding members 3a, 3b. The shared holding member 4 may be made of a rigid material, which is not easily deformed elastically when force is executed onto it.

Two drug delivery devices 6a, 6b can be releasably secured between the holding members 3a, 3b and 4. For distinguishing between the drug delivery devices 6a, 6b (not shown in FIG. 3) retained by the clip elements 2a, 2b, the outer holding members 3a, 3b of the clip elements 2a, 2b may comprise different colours. In particular, holding member 4, which is shared by the two clip elements 2a, 2b, does not have to comprise a dedicated colour.

In this embodiment the two clip elements 2a, 2b comprise a different size, accounting for two different drug delivery devices 6a, 6b (not shown in FIG. 3) to be secured to the fixing means 1. In addition, the two clip elements 2a, 2b may again comprise—apart from different colours—differently marked surfaces, indicated by the knobs 11 on the surface of one clip element 2b, for facilitating the differentiation of the two drug delivery devices 6a, 6b to be received between the holding members 3a, 3b and 4 of the clip elements 2a, 2b.

Holding members 3a, 3b and 4 may have an elongated shape, thereby defining an elongated holding space. Accordingly, rotational movement of the drug delivery device 6a, 6b with respect to the holding members 3a, 3b and 4 may be prevented more easily.

FIG. 4 schematically shows an embodiment of a carry case containing the fixing means of FIG. 2 on the basis of an oblique partial view.

FIG. 4 represents the fixing means 1 of FIG. 2 having the two clip elements 2a, 2b. In addition, FIG. 4 shows a carry case 7 and a retaining means 8. The carry case 7 comprises a cover 12, a circumferential wall 13, a bottom 14 and a zip 15 as well as a rubber band 17.

For providing protection of the fixing means 1 and, above all, for providing additional protection of the drug delivery devices 6a, 6b from vibrations and environmental influences, the fixing means 1 may be secured to the carry case 7 by means of attaching means 5, as already explained in connection with FIG. 1.

The carry case 7 may be made of a robust and water-repellent material. Preferably, the carry case 7 is closeable. For closing the carry case 7, it may provide a zip 15, as indicated by FIG. 4, or one or more snap-fasteners.

In this embodiment the carry case 7 comprises cover 12 and bottom 14. Bottom 14 is surrounded by the circumferential wall 13. When the fixing means 1 is attached to the carry case 7, i.e. preferably on the bottom 14 of the carry case 7, a user may move the cover 12 towards the bottom 14 until the cover 12 abuts the wall 13. The user may thus close the carry case 7 and in particular, secure the cover 12 to the bottom 14, for example via zip 15.

The fixing means 1 may be glued to the inside of said carry case 7. Accordingly or alternatively, the fixing means 1 may comprise the attaching means 5, for example cut-outs, for securing the fixing means 1 to mating engaging means, for example protrusions (not shown in FIG. 4), of the carry case 7. Alternatively or additionally, the fixing means 1 may be sewed to the carry case 7 as mentioned in connection with the description of FIG. 1. Alternatively or additionally, the fixing means 1 may be secured to the carry case 8 by means of a velcro connection.

After having secured one or more drug delivery devices 6a, 6b to the fixing means 1, a user can lock the carry case by means of the zip 15 and/or snap-fasteners, hence protecting the drug delivery devices 6a, 6b and giving rise to an easy and safe storage and/or transport of the drug delivery devices 6a, 6b.

The carry case 7 comprises in this embodiment the retaining means 8 at the inside of the cover 12, which is configured to retain information about the drug delivery devices 6a, 6b secured to the fixing means 1. The retained information may help the user to determine, for example, the right dose of a drug and/or the intervals at which a dose has to be dispensed. Accordingly or alternatively, the information retained can be user-related information, for example, the name and/or the address of the user, so that the carry case 7 and hence, the drug delivery device, can be returned to the user if the user has lost the carry case 7. The retaining means 8 may be configured for the transport of an additional and/or a replacement cartridge.

The retaining means 8 may be a pocket within the carry case 7, made of the same material as the carry case 7. Accordingly or alternatively, the retaining means 8 may be made of a transparent material, for example plastic, so that a user can see at once whether the retaining means 8 holds information and what information is retained. Preferably, the pocket is glued or sewed to the inside, i.e. the inside of the cover 12 or the bottom 14, of the carry case 7. Accordingly or alternatively, the retaining means 8 may comprise a rigid box, which is preferably glued to the inside, i.e. the inside of the cover 12 or the bottom 14, of the carry case 7. Accordingly or alternatively, the retaining means 8 may comprise a meshed pocket (not shown in FIG. 4), which may be closeable by means of zip 15, for preventing the information or elements retained in the retaining means 8 from sliding out of said retaining means 8.

In addition, the rubber band 17 may be arranged at the inside of the cover 12 of the carry case 7. Said rubber band 17 may extend transversally with respect to a longitudinal direction of main extent of the cover 12 of the carry case 7 and may be configured to retain, for example, further information concerning the drug delivery device 1. The rubber band 17 may be configured to retain a replacement needle and/or a replacement cartridge.

FIG. 5 schematically shows an embodiment of a drug delivery device which may be secured to a fixing means according to any of the previously described embodiments on the basis of a sectional view.

FIG. 5 shows a drug delivery device 6 comprising a housing 18 and a cartridge 19. The cartridge retains a drug 20, for example heparin, insulin, like short-acting or long-acting insulin, or growth hormones. The term “drug”, as used herein, preferably means a pharmaceutical formulation containing at least one pharmaceutically active compound, wherein in one embodiment the pharmaceutically active compound has a molecular weight up to 1500 Da and/or is a peptide, a proteine, a polysaccharide, a vaccine, a DNA, a RNA, a antibody, an enzyme, an antibody, a hormone or an oligonucleotide, or a mixture of the above-mentioned pharmaceutically active compound.

In a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis.

In a further embodiment the pharmaceutically active compound comprises at least one peptide for the treatment and/or prophylaxis of diabetes mellitus or complications associated with diabetes mellitus such as diabetic retinopathy.

In a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-γ-glutamyl)-des(B30) human insulin; B29-N—(N-lithocholyl-γ-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

### H-(Lys)4-des Pro36, des Pro37 Exendin-4(1-39)-NH2,

### H-(Lys)5-des Pro36, des Pro37 Exendin-4(1-39)-NH2,

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39); or

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39),

wherein the group-Lys6-NH2 may be bound to the C-terminus of the Exendin-4 derivative;
or an Exendin-4 derivative of the sequence

### H-(Lys)6-des Pro36 [Asp28] Exendin-4(1-39)-Lys6-NH2,

### des Asp28 Pro36, Pro37, Pro38Exendin-4(1-39)-NH2,

### H-(Lys)6-des Pro36, Pro38 [Asp28] Exendin-4(1-39)-NH2,

### H-Asn-(Glu)5des Pro36, Pro37, Pro38 [Asp28] Exendin-4(1-39)-NH2,

### des Pro36, Pro37, Pro38 [Asp28] Exendin-4(1-39)-(Lys)6-NH2,

### H-(Lys)6-des Pro36, Pro37, Pro38 [Asp28] Exendin-4(1-39)-(Lys)6-NH2,

### H-Asn-(Glu)5-des Pro36, Pro37, Pro38 [Asp28] Exendin-4(1-39)-(Lys)6-NH2,

### H-(Lys)6-des Pro36 [Trp(O2)25, Asp28] Exendin-4(1-39)-Lys6-NH2,

### H-des Asp28 Pro36, Pro37, Pro38 [Trp(O2)25] Exendin-4(1-39)-NH2,

### H-(Lys)6-des Pro36, Pro37, Pro38 [Trp(O2)25, Asp28] Exendin-4(1-39)-NH2,

### H-Asn-(Glu)5-des Pro36, Pro37, Pro38 [Trp(O2)25, Asp28] Exendin-4(1-39)-NH2,

### des Pro36, Pro37, Pro38 [Trp(O2)25, Asp28] Exendin-4(1-39)-(Lys)6-NH2,

### H-(Lys)6-des Pro36, Pro37, Pro38 [Trp(O2)25, Asp28] Exendin-4(1-39)-(Lys)6-NH2,

### H-Asn-(Glu)5-des Pro36, Pro37, Pro38 [Trp(O2)25, Asp28] Exendin-4(1-39)-(Lys)6-NH2,

### H-(Lys)6-des Pro36 [Met(O)14, Asp28] Exendin-4(1-39)-Lys6-NH2,

### des Met(O)14 Asp28 Pro36, Pro37, Pro38 Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Asp28] Exendin-4(1-39)-NH2,

### des Pro36, Pro37, Pro38 [Met(O)14, Asp28] Exendin-4(1-39)-(Lys)6-NH2,

### H-(Lys)6-des Pro36, Pro37, Pro38 [Met(O)14, Asp28] Exendin-4(1-39)-(Lys)6-NH2,

### H-Asn-(Glu)5 des Pro36, Pro37, Pro38 [Met(O)14, Asp28] Exendin-4(1-39)-(Lys)6-NH2,

### H-Lys6-des Pro36 [Met(O)14, Trp(O2)25, Asp28] Exendin-4(1-39)-Lys6-NH2,

### H-des Asp28 Pro36, Pro37, Pro38 [Met(O)14, Trp(O2)25] Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Trp(O2)25, Asp28] Exendin-4(1-39)-NH2,

### des Pro36, Pro37, Pro38 [Met(O)14, Trp(O2)25, Asp28] Exendin-4(1-39)-(Lys)6-NH2,

### H-(Lys)6-des Pro36, Pro37, Pro38 [Met(O)14, Trp(O2)25, Asp28] Exendin-4(1-39)-(Lys)6-NH2,

### H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na⁺, or K⁺, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

The drug delivery device 6 further comprises a dose member 21, a plunger 22 and a plunger seal 23. The device 6 has a distal end and a proximal end. The distal end is indicated by the arrow 24, which refers to that end of the drug delivery device 6 which is closest to a dispensing end of the drug delivery device 6. The proximal end is indicated by the arrow 25 referring to that end which is furthest away from the dispensing end of the device 6.

The drug delivery device 6 is a pen-type device, in particular a pen-type injector. The device 6 may be a disposable or a reusable device and may be configured to dispense fixed doses of the drug 20 or variable, preferably user-settable doses of the drug 20.

The drug delivery device 6 may further comprise a needle assembly (not shown in FIG. 5) at the distal end of the drug delivery device 6, which assembly may comprise a needle and a needle retainer preventing a displacement of the needle with respect to the needle retainer during dose delivery. Alternatively, the drug delivery device 6 may be a needle-free device.

The drug delivery device 6 comprises an elongated shape which facilitates reliably securing the device 6 to the fixing means 1 according to one of the above described embodiments of the fixing means 1.

Other implementations are within the scope of the following claims. Elements of different implementations may be combined to form implementations not specifically described herein.

### REFERENCE NUMERALS

1 Fixing means
2 Clip element
2a Clip element
2b Clip element
3 Holding member
3a Holding member
3b Holding member
4 Holding member
5 Attaching means
6 Drug delivery device
6a Drug delivery device
6b Drug delivery device
7 Carry case
8 Retaining means
9 Bearing surface
10 Bulge
10a Bulge
10b Bulge
11 Knob
12 Cover
13 Wall
14 Bottom
15 Zip
16 Free end
16a Free end
16b Free end
17 Rubber band
18 Housing
19 Cartridge
20 Drug
21 Dose member
22 Plunger
23 Plunger seal
24 Distal end
25 Proximal end

1. A fixing means for a drug delivery device, wherein the fixing means comprises at least two clip elements for releasably securing at least one drug delivery device to said fixing means, wherein each of the two clip elements comprises a flexible holding member and wherein the clip elements share one flexible holding member with each other.

2. The fixing means of claim 1, wherein the clip element is configured to encompass the at least one drug delivery device.

3. The fixing means of claim 1, wherein the holding members are arranged to receive and retain the at least one drug delivery device in a holding space extending between each other.

4. The fixing means according to claim 1, wherein the holding member of the clip element is configured to bow radially outwardly with respect to the at least one drug delivery device while introducing the device into or releasing the device from the fixing means.

5. The fixing means according to claim, wherein the holding members comprise free ends and wherein the distance between the free ends of the holding members is smaller than the diameter of the drug delivery device.

6. The fixing means according to claim 1, wherein the two clip elements are marked differently.

7. The fixing means according to claim 1, wherein the fixing means comprises at least one attaching means for attaching the fixing means to a carry case.

8. The fixing means according to claim 7, wherein the attaching means comprises at least one cut-out.

9. The fixing means according to claim 1, wherein the at least one drug delivery device is a pen-type drug delivery device.

10. An assembly comprising a drug delivery device secured to the fixing means of claim 1.

11. A carry case comprising the fixing means according to claim 1, wherein the fixing means is attached to the carry case and wherein the carry case comprises a retaining means for retaining information about the at least one drug delivery device and/or for retaining information related to a user of said drug delivery device.
